(19) United States
(12) Patent Application Publication
(10) Pub. No.: US 2005/0065141 A1
(43) Pub. Date: Mar. 24, 2005

(54) Carbapenems useful in treating and preventing pulmonary infections, pharmaceutical compositions thereof and modes of administration thereof

(76) Inventors: Debra A. Odink, Oakland, CA (US); Paul F. Truex, Pleasanton, CA (US); Yigong Ge, South San Francisco, CA (US)

Correspondence Address: Ann M. Caviani Pease/Sunil K. Singh;Dorsey & Whitney LLP, Intellectual Property Department, Four Embarcadero Center, Suite 3400, San Francisco, CA, 94111-4187, US

(21) Appl. No.: 10/910,243

(22) Filed: Aug. 2, 2004

Related U.S. Application Data
(60) Provisional application No. 60/491,912, filed on Jul. 31, 2003.

(51) Int. Cl. A61K 31/407

(52) U.S. Cl. 514/210.09

(57) ABSTRACT

The present invention provides carbapenems to treat or prevent pulmonary infections, particularly in patients with cystic fibrosis, pneumonia, ventilator associated pneumonia, bronchitis or bronchiectstasis, pharmaceutical compositions of these carbapenems and methods of administering these carbapenems to treat or prevent pulmonary infections.

### 1. FIELD

The present invention relates generally to the use of carbapenems to treat or prevent pulmonary infections, particularly in patients with cystic fibrosis, pneumonia, ventilator associated pneumonia, bronchitis or bronchiectstasis, pharmaceutical compositions of these carbapenems and methods of administering these carbapenems and/or pharmaceutical compositions thereof to treat or prevent pulmonary infections.

### 2. BACKGROUND

Gram negative and gram positive bacteria are responsible for a wide variety of severe pulmonary infections. Many of the above bacteria are refractory to treatment with conventional orally administered antibiotics. Accordingly, respiratory infections caused by bacteria resistant to antibiotic therapy is a severe problem in patients with reduced immune function (e.g., patients with cystic fibrosis, infected with HIV, suffering from autoimmune disorders, etc.), bronchiectasis and pneumonia, particularly, ventilator associated pneumonia.

Currently, most pulmonary infections are treated with parenterally administered antibiotics, which are often used in two to three way combination since many antibiotics are not effective against both gram positive bacteria and gram negative bacteria. For example, tobramycin, an aminoglycoside antibiotic is effective against only gram positive bacteria, while aztreonam is effective only against gram negative bacteria (Montgomery, U.S. Pat. No. 6,083,922; Montgomery U.S. patent application Ser. No. 2003/0055034). In many situations, a therapeutically effective dose of an antibiotic, particularly one that is delivered parenterally, often has an unacceptable therapeutic index (i.e., the required dose is often toxic).

Carbapenems, which are β-lactam antibiotics, have been widely used to treat bacterial infection since the discovery of imipenem (i.e., N-formimidoyl thienamycin). Importantly, carbapenems are typically broad spectrum antibiotics active against gram positive, gram negative, anaerobic and aerobic bacteria which are also fairly resistant to hydrolysis by β-lactamases.

Accordingly, there is a need to develop new methods of treating pulmonary infections, particularly new methods of treating pulmonary infections with carbapenems. Preferably, these new methods will include formulating carbapenems for direct delivery to the lung and methods for delivering carbapenems to the lung.

### 3. SUMMARY

The present invention satisfies these and other needs by providing carbapenems to treat or prevent pulmonary infections, particularly in patients with cystic fibrosis, pneumonia, ventilator associated pneumonia, bronchitis or bronchiectstasis, pharmaceutical compositions of these carbapenems and methods of administering these carbapenems and/or pharmaceutical compositions thereof to treat or prevent pulmonary infections.

In one aspect, the present invention provides a method of treating or preventing pulmonary infection in a patient in need of such treatment. A therapeutically effective amount of a carbapenem or a pharmaceutically acceptable salt, hydrate, solvate or pro-drug thereof is administered to the patient. In one embodiment, the patient has cystic fibrosis, pneumonia, ventilator associated pneumonia, bronchitis or bronchiectstasis. In another embodiment, the carbapenem is imipenem, meropenem, faropenem, biapenem, ertapenem, panipenem, ritipenem or sulopenem. In still another embodiment, the carbapenem is a pyrrolidylthiocarbapenem of Formula (I): 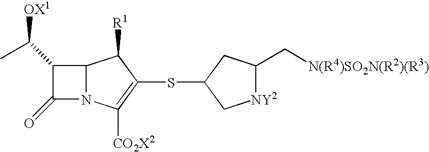
wherein:

R¹ is hydrogen or lower alkyl;

R², R³, R⁴ are independently hydrogen, lower alkyl, substituted lower alkyl, an amino protecting group or R² and R³ together with the nitrogen atom with which they are bonded form a saturated or unsaturated cyclic group or R² and R⁴ or R³ and R⁴ together with two nitrogen atoms and one sulfur atom in the sulfamide group form a saturated or unsaturated cyclic group; each cyclic group can further include at least one atom selected from the group consisting of oxygen, sulfur and nitrogen and each cyclic group can be substituted;

X¹ is hydrogen or a hydroxy protecting group;

X² is hydrogen, a carboxy protecting group, an ammonio group, an alkali metal or an alkaline earth metal; and

Y² is hydrogen or an amino protecting group.

Preferably, the therapeutically effective amount of the carbapenem or pharmaceutically acceptable salt, hydrate, solvate or pro-drug thereof is between about 5 mg and about 2000 mg, more preferably, between about 25 mg and about 500 mg. Preferably, the therapeutically effective amount of doripenem or pharmaceutically acceptable salt, hydrate, solvate or pro-drug thereof is between about 20 mg and about 1000 mg, more preferably, between about 50 mg and about 300 mg.

In other embodiments, the therapeutically effective amount of carbapenem or pharmaceutically acceptable salt, hydrate, solvate or pro-drug thereof is administered by inhalation into the lung of the patient. Preferably, the therapeutically effective amount of carbapenem or pharmaceutically acceptable salt, hydrate, solvate or pro-drug thereof is administered with an electrohydrodynamic aerosol device, an aerosol device, a dry powder inhaler, a multi dose dry powder inhaler or a nebulizer device, more preferably, with a nebulizer device.

In some other embodiments, the pulmonary infection is caused by both gram negative and gram positive bacteria. In still other embodiments, the pulmonary infection is caused by Psuedomonas aeruginosa, Staphylococcus aureaus, Haemophilus influenzae, Burkholderia cepacia, streptococci, Staphylococci (methicillin susceptible), Enterobacteriaceae, Moraxella catarrhalis, Bacteroides spp., Clostridium spp., Peptostreptococcus spp. and Neisseria spp.

In another aspect, the present invention provides pharmaceutical compositions suitable for treating a patient suffering from a pulmonary infection. The pharmaceutical compositions comprise an amount of a carbapenem or a pharmaceutically acceptable salt, hydrate, solvate or prodrug thereof and a pharmaceutically acceptable vehicle where the amount is sufficient to treat the pulmonary infection. In some embodiments, the pharmaceutically acceptable vehicle is a liquid. In other embodiments, the pharmaceutically acceptable vehicle is water, saline, alcohol, surfactant or perfluorocarbon. In still other embodiments, the pharmaceutically acceptable vehicle is saline.

In still other embodiments, between about 5 mg to about 250 mg of the carbapenem or pharmaceutically acceptable salt, hydrate, solvate or prodrug thereof is dissolved in between about 1 mL to about 10 mL of between about a 0.1% and 0.9% saline solution. Preferably, the pH is between about 4.5 to about 7.5, more preferably, between about 5.0 to about 6.5.

In still another aspect, the present invention provides methods of treating a pulmonary infection in a patient in need of such treatment. A pharmaceutical composition comprising a carbapenem and a pharmaceutically acceptable vehicle is administered to the patient. In some embodiments, the pharmaceutical composition is administered by inhalation into the lung of the patient.

In other embodiments, the pharmaceutical composition is administered by a nebulizer device into the lung of the patient. Preferably, the nebulizer is a jet, electronic, ultrasonic or atomization nebulizer. In still other embodiments, the pharmaceutical composition forms an aerosol of particles with mass median aerodynamic diameter of between about 1.0 μm to about 5.0 μm.

In still another aspect, the present invention provides pharmaceutical compositions suitable for preventing pulmonary infection in a patient at risk of a pulmonary infection. An amount of a carbapenem or a pharmaceutically acceptable salt, hydrate, solvate or prodrug thereof and a pharmaceutically acceptable vehicle is administered to a patient, said amount being sufficient to prevent the pulmonary infection.

In still another aspect, the present invention provides a method of treating a pulmonary infection in a patient at risk of a pulmonary infection. A pharmaceutical composition comprising a carbapenem and a pharmaceutically acceptable vehicle is administered to the patient.

In still another aspect, the current invention provides a therapeutic kit. In some embodiments, the therapeutic kit has a first container containing a carbapenem or pharmaceutically acceptable salt, hydrate, solvate or prodrug thereof as a dry powder and a second container containing a pharmaceutically acceptable vehicle. In some embodiments, the carbapenem is doripenem and the pharmaceutically acceptable vehicle is saline.

### 4. DETAILED DESCRIPTION

### 4.1 Definitions

“Patient” includes humans. The terms “human” and “patient” are used interchangeably herein.

“Pharmaceutically acceptable salt” refers to a salt of a compound of the invention, which is pharmaceutically acceptable and possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a carbapenem is administered.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### 4.2 Carbapenems

“Carbapenem” refers to compounds which include the carbapenem nucleus shown below as part of their chemical structure.

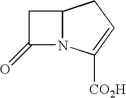

Accordingly, those of skill in the art will appreciate the term “carbapenem” includes all possible compounds where any of the hydrogen atoms in the carbapenem nucleus have been substituted by either organic or inorganic groups. Examples of carbapenems include, but are not limited to, imipenem, meropenem, faropenem, biapenem, ertapenem, panipenem, ritipenem, sulopenem doripenem, etc.

In one embodiment, a carbapenem useful in the present invention is represented by Formula I below: 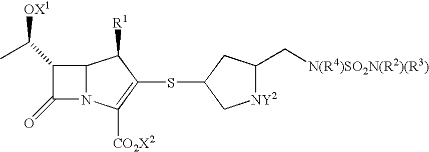
wherein R¹ is hydrogen or lower alkyl; R², R³ and R⁴ are hydrogen, lower alkyl which can be substituted or an amino protecting group independently and preferably R⁴ is hydrogen, or R² and R³ together with a nitrogen atom to which R² and R³ are bonded form a saturated or unsaturated cyclic group, or R² and R⁴, or R³ and R⁴ together with two nitrogen atoms and one sulfur atom in the sulfamide group form a saturated or unsaturated cyclic group and each cyclic group can further include at least one atom selected from the group consisting of oxygen, sulfur and nitrogen, and each cyclic group can be substituted; X¹ is hydrogen or a hydroxy protecting group; X² is hydrogen, a carboxy protecting group, an ammonio group, an alkali metal or an alkaline-earth metal; and Y² is hydrogen or an amino protecting group. In some embodiments, R¹ is methyl, R², R³, R⁴, X¹ and Y² are hydrogen and X² is hydrogen or an alkali metal.

The number of carbon atoms of “lower alkyl” is 1 to 6. Examples of such an alkyl group include methyl, ethyl, n-propyl, iso-propyl, n-butyl, t-butyl, pentyl and hexyl. The number of carbon atoms of the lower alkyl is preferably 1 to 4. The most preferred lower alkyl is methyl or ethyl. Examples of a substituent of “a substituted lower alkyl” include hydroxy, alkoxy, amino, acylamino, lower alkylamino, carbamoyl, lower carbamoyl, lower alkylcarbamoyloxy and cyano. The number of carbon atoms of “aralkyl” is 7 to 15. Examples of “an amino protecting group” and “a hydroxy protecting group” include lower alkoxycarbonyl, lower alkenyloxycarbonyl, halogenoalkoxycarbonyl, aralkyloxycarbonyl, trialkylsilyl and diazo. An example of lower alkoxycarbonyl includes t-butyloxycarbonyl; an example of lower alkenyloxycarbonyl includes allyloxycarbonyl; examples of halogenoalkoxycarbonyl include 2-iodoethyloxycarbonyl and 2,2,2-trichloroethylozycarbonyl; examples of the aralkyloxycarbonyl include benzyloxycarbonyl, p-methoxybenzyloxycarbonyl, o-nitrobenzyloxycarbonyl, p-nitrobenzyloxycorbonyl and diphenylmethoxycarbonyl; examples of trialkylsilyl include trimethylsilyl, triethylsilyl and t-butyldimethylsilyl.

In a definition of a group represented as follows: 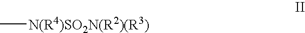
a saturated or unsaturated cyclic group formed from R² and R³ together with a nitrogen atom to which R² and R³ are bonded can be, a saturated or unsaturated 3 to 8 membered residue further having one or more of nitrogen, sulfur and/or oxygen atoms, if necessary, and a 5 or 6 membered monocyclic residue including a hetero atom is preferable. The examples include pyrrolidinl-yl, pyrrol-1-yl, imidazolidin-1-yl, imidazol-1-yl, pyrazolidin-1-yl, pyrazol-1-yl, piperidino, dihydro- or tetrahydropyridin-1-yl, piperazino, piperazin-1-yl which may have a substituent at the 4-position, morpholino and thiomorpholino. These groups may be substituted for one or more, preferably one or two of the following groups: amino, protected amino, carbamoyl, lower alkyl, hydroxy, protected hydroxyl, lower alkoxy, oxo, lower alkylsulfonyl, hydroxy lower alkyl, carbamoyl lower alkyl, lower alkoxycarbonyl and cyano. Moreover, when the cyclic group is imidazolidin 1-yl, pyrazolidin-1-yl or piperazin-1-yi, the imino moiety thereof may be protected by an imino protecting group which is known in the art.

In the definition of the group II, a saturated or unsaturated cyclic group formed from R² and R⁴, or R³ and R⁴ can be a saturated or unsaturated 5 to 7 membered residue having 2 to 3 nitrogen atoms and one sulfur atom and if necessary, having an another hetero atom such as an oxygen atom, and 5 to 6 membered monocyclic residue including a hetero atom is preferable. Such a residue may include, if necessary, a substituent such as lower alkyl, halogen, lower alkoxy, acyloxy, hydroxy, amino, lower alkylamino, acylamino and oxo, and/or an unsaturated bond. The examples include 1,1-dioxothiadiazinyl, 1,1-dioxodihydrothiadiazinyl, 1,1,3-trioxodihydrothiadiazinyl, 1,1-dioxothiadiazolizinyl, 1,1dioxothiadiazolinyl, and 1,1,3-trioxothiadiazolinyl.

The “carboxy protecting group” is selected from those used in the art and serves the function of blocking the carboxyl group while reactions are carried out at other sites of the molecule. Such a group generally contains less than about 19 carbon atoms and binds to a carboxyl group reversibly without affecting the other parts of the molecule. Typical examples include following groups: optionally substituted C¹-C⁸ alkyl, for example, methyl, methoxymethyl, ethyl, ethoxymethyl, iodomethyl, propyl, isopropyl, butyl, isobutyl, ethoxyethyl, methylthioethyl, methanesulfonylethyl, trichloroethyl, t-butyl and the like; optionally substituted C³-C⁸ alkenyl, for example, propenyl, allyl, isoprenyl, hexenyl, phenylpropenyl, dimethylhexenyl, and the like; optionally substituted C⁷-C¹⁹ aralkyl, for example, benzyl, methylbenzyl, dimethylbenzyl, methoxybenzyl, ethoxybenzyl, nitrobenzyl, aminobenzyl, diphenylmethyl, phenylethyl, trityl, di-t-butylhydroxybenzyl, phthalidyl, phenacyl, and the like; optionally substituted C⁶-C¹² aryl, for example, phenyl, toluyl, diisopropylphenyl, xylyl, trichlorophenyl, pentachlorophenyl, indanyl, and the like; optionally substituted C¹-C¹² amino which is, e.g., an ester with acetone oxime, acetophenone oxime, acetoaldoxime, N-hydroxysuccinimide, N-hydroxyphthalimide, or the like; optionally substituted C³-C¹² hydrocarbonated silyl, for example, trimethylsilyl, dimethylmethoxysiIyl, t-butyldimethylsilyl and the like; optionally substituted C³-C¹² hydrocarbonated stannyl, for example, trimethylstannyl and the like. Another carboxy protecting group is a pharmaceutically active ester forming group. Examples of such a group include following groups: 1-(oxygen substituted)-C² to C¹⁵ alkyl groups, for example, a straight, branched, ringed, or partially ringed alkanoyloxyalkyl, such as acetoxymethyl, acetoxyethyl, propionyloxymethyl, pivaloyloxymethyl, pivaloyloxyethyl, cyclohexaneacetoxyethyl, cyclohexanecarbonyloxycyclohexylmethyl, and the like; C³-C¹⁵ alkoxycarbonyloxyalkyl such as ethoxycabonyloxyethyl, and the like; C²-C⁸ alkoxyalkyl, such as methoxymethyl, methoxyethyl and the like; C⁴-C⁸ 2-oxacycloalkyls, such as tetrahydropyranyl, tetrahydrofuranyl, and the like; substituted C³-C¹² aralkyls, for example, phenacyl, phthalidyl, and the like; C⁶-C¹² aryl, for example, phenyl, xylyl, indanyl and the like; C²-C¹² alkenyl, for example, allyl, isoprenyl, 2-oxo-1,3-dioxolyl-4-ylmethyl, and the like. Among the above, a protecting group used to block the carboxyl group during reactions is usually removed at the final step of the reaction, and therefore its structure is not essential. Thus, as one skilled in the art can easily appreciate, the carboxy protecting group can be selected from various equivalent groups including amides, acid anhydrides formed with carbonic acid or carboxylic acids and the like.

An example of lower alkyl includes t-butyl; examples of lower alkenyl includes allyl, isopentenyl and 2-butenyl; examples of halogeno lower alkyl include 2-iodoethyl and 2,2,2-trichloroethyl; examples of the lower alkoxymethyl include methoxymethyl, ethoxymethyl and isobutoxymethyl; examples of lower aliphatic acyloxymethyl include acetoxymethyl, propionyloxymethyl, butyryloxymethyl and pivaloyloxymethyl; examples of lower alkoxycarbonyloxyethyl include 1-methoxycarbonyloxyethyl and 1-ethoxycarbonyloxyethyl; and examples of aralkyl includes benzyl, p-methoxybenzyl, o-nitrobenzyl, p-nitrobenzyl and diphenylmethyl. Examples of “an alkali metal” include lithium, sodium and potassium, and sodium or potassium is preferred. Examples of “an alkaline-earth metal” include magnesium and calcium.

As “a mercapto protecting group”, a conventional one, e.g., acyl and aryl substituted lower alkyl such as benzyl, phenethyl, trityl and benzhydryl are included. As “a reactive ester group of hydroxy”, a conventional one, e.g., a residue such as substituted or unsubstituted arylsulfonyloxy, lower alkanesulfonyloxy, halogeno lower alkanesulfonyloxy, dialkylphosphonyloxy, diarylphosphoryloxy and halogeno are included. Examples of the arylsulfonyloxy include benzenesulfonyloxy, p-toluenesulfonyloxy, p-nitrobenzenesulfonyloxy and p-bromobenzenesulfonyloxy; examples of the lower alkanesulfonyloxy include methanesulfonyloxy and ethanesulfonyloxy; an example of the halogeno lower alkanesulfonyloxy includes trifluoromethanesulfonyloxy; an example of the dialkylphosphoryloxy includes diethylphosphoryloxy; an example of the diarylphosporyloxy includes diphenylposphoryloxy; and examples of the halogeno include chloro, bromo and iodo.

An example of “an alkylsulfinyl group” includes methylsulfinyl, and an example of “an arylsulfinyl group” includes phenylsulfinyl.

Carbapenems may be identified either by chemical structure and/or chemical name. When the chemical structure and chemical name conflict, the chemical structure is determinative of the identity of the compound. Carbapenems may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Carbapenems may also exist in several tautomeric forms. Accordingly, the chemical structures depicted herein encompass all possible tautomeric forms of the illustrated compounds. Carbapenems also include isotopically labeled compounds where one or more atoms have an atomic mass different from the atomic mass conventionally found in nature. Examples of isotopes that may be incorporated into the compounds of the invention include, but are not limited to, ²H, ³H, ¹¹C, ¹³C, ¹⁴C, ¹⁵N, ¹⁷O, ¹⁸O, etc. Carbapenems may exist in unsolvated forms as well as solvated forms, including hydrated forms and as N-oxides. In general, the hydrated, solvated and N-oxide forms are within the scope of the present invention. Certain carbapenems may exist in multiple crystalline or amorphous forms. In general, all physical forms are equivalent for the uses contemplated by the present invention and are intended to be within the scope of the present invention.

### 4.3 Carbapenem Synthesis

Carbapenems (e.g., imipenem, meropenem, faropenem, biapenem, ertapenem, panipenem, ritipenem, sulopenem, etc.) may be obtained via either known synthetic routes or conventional synthetic methods both of which are known to those of skill in the art. Starting materials useful for preparing carbapenems and intermediates thereof are commercially available or can be prepared by well-known synthetic methods. Doripenem and doripenem derivatives, may be obtained by methods described in the art (Nishitani et al., U.S. Pat. No. 5,317,016; Nishitani et al., U.S. Pat. No. 5,703,243; Sendo et al., U.S. Pat. No. 5,539,102).

### 4.4 Therapeutic Uses

In accordance with the invention, a carbapenem and/or a pharmaceutical composition thereof is administered to a patient, preferably a human, suffering from a pulmonary infection. Pulmonary infections include, but are not limited to, infections caused by Escheria coli, Klebsiella pneumoniae, Klebsiella oxytoca, Proteus mirablis, Entrebacter species, Serratia marcescens, Stenotrophomoas maltophilia, Alcaligenes xylosoxidans, Psuedomonas aeruginosa, Staphylococcus aureaus, Haemophilus influenzae, Burkholderia cepacia, staphylococci Staphylococci (methicillin susceptible), Enterobacteriaceae, Moraxella catarrhalis, Bacteroides spp., Clostridium spp., Peptostreptococcus spp. and Neisseria spp.

Further, in certain embodiments, carbapenems and/or pharmaceutical compositions thereof are administered to a patient, preferably a human, as a preventative measure against pulmonary infections. Thus, carbapenems and/or pharmaceutical compositions thereof may be administered as a preventative measure to a patient having a predisposition for a pulmonary infection (e.g., a patient suffering from cystic fibrosis). Accordingly, the carbapenems and/or pharmaceutical compositions thereof may be used for the prevention of type of pulmonary infection while concurrently treating another (e.g., preventing Psuedomonas aeruginosa pulmonary infection while treating Burkholderia cepacia pulmonary infection).

The suitability of carbapenems and/or pharmaceutical compositions thereof in treating or preventing pulmonary infections may be assayed by methods described below and in the art. Accordingly, it is well with the capability of those of skill in the art to assay and use the compounds and/or pharmaceutical compositions of the invention to treat or prevent diseases or disorders characterized by pulmonary infections.

Generally, active carbapenems may be identified using in vitro screening assays. Additionally, despite certain apparent limitations of in vitro susceptibility tests, clinical data indicate that a good correlation exists between minimal inhibitory concentration (MIC) test results and in vivo efficacy of antibiotic compounds (Murray, 1994, Antimicrobial Susceptibility Testing, Poupard et al., eds., Plenum Press, NY; Knudsen et al., 1995, Antimicrob. Agents Chemother. 39 (6):1253-1258). Thus, carbapenems useful in the current invention are conveniently identified by demonstrated in vitro antimicrobial activity against specific microbial targets found in common pulmonary infections.

Generally, in vitro antimicrobial activity of antimicrobial agents may be tested using standard NCCLS bacterial inhibition assays, or MIC tests (e.g., National Committee on Clinical Laboratory Standards “Performance Standards for Antimicrobial Susceptibility Testing,” NCCLS Document M100-S5 Vol. 14, No. 16, December 1994; “Methods for dilution antimicrobial susceptibility test for bacteria that grow aerobically-Third Edition,” Approved Standard M7-A3, National Committee for Clinical Standards, Villanova, Pa.).

Alternatively, carbapenems may be assessed for antimicrobial activity using in vivo models. Again, such models are well-known in the art.

It will be appreciated that other assays, that are well known in the art or which will become apparent to those having skill in the art upon review of this disclosure, may also be used to identify active carbapenems, which may be used in the current invention. Such assays include, for example, the assay described in Lehrer et al., 1988, J. Immunol. Methods 108:153 and Steinberg et al., “Designer Assays for Antimicrobial Peptides: Disputing the ‘One Size Fits All’ Theory,” In: Antibacterial Peptide Protocols, Shafer, Ed., Humana Press, N.J.

Generally, carbapenems useful in the current invention will exhibit MICs of less than about 64 g/mL, usually less than about 32 g/mL, preferably less than about 16 g/mL and most preferably less than about 4 g/mL.

Of course, compounds having MICs on the low end of these ranges, or even lower, are preferred. Most preferred for use in treating or preventing pulmonary infections are carbapenems that exhibit significant antimicrobial activity (i.e., less than 4 g/mL), good water-solubility (at approx. between pH 4.5 to 7.0) and low toxicity.

### 4.5 Therapeutic/Prophylactic Administration

Carbapenems and/or pharmaceutical compositions thereof may be advantageously used in human medicine. As previously described, supra, carbapenems and/or pharmaceutical compositions thereof are useful for the treatment or prevention of various diseases or disorders characterized by pulmonary infections.

When used to treat or prevent the above disease or disorders, carbapenems and/or pharmaceutical compositions thereof may be administered or applied singly, or in combination with other agents. Carbapenems and/or pharmaceutical compositions thereof may also be administered or applied singly, in combination with other pharmaceutically active agents including other carbapenems.

The current invention provides methods of treatment and prophylaxis by administration to a patient of a therapeutically effective amount of a carbapenem and/or pharmaceutical composition thereof. The patient may be an animal, is more preferably, a mammal and most preferably, a human.

In one embodiment, one or more carbapenems and/or pharmaceutical composition thereof is administered locally to the lungs of a patient. A carbapenem and/or pharmaceutical composition thereof may be administered to the lung by inhalation. For administration by inhalation, carbapenems may be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas may be used to deliver carbapenems directly to the lung. MDI devices are available from a number of suppliers such as Aventis (Bridgewater, N.J.) and Vectura, Ltd (Chippenham, UK).

Alternatively, a Dry Powder Inhaler (“DPI”) device may be used to administer carbapenems to the lung. DPI devices typically use a mechanism such as a burst of gas to create a cloud of dry powder inside a container, which may then be inhaled by the patient. DPI devices are also well known in the art and may be purchased from a number of vendors which include, for example, Nektar Therapeutics (San Carlos, Calif.) and Vectura, Ltd (Chippenham, UK). A popular variation is the multiple dose DPI (“MDDPI”) system, which allows for the delivery of more than one therapeutic dose. MDDPI devices are available from companies such as Vectura, Ltd (Chippenham, UK). For example, capsules and cartridges of gelatin for use in an inhaler or insufflator may be formulated containing a powder mix of the compound and a suitable powder base such as lactose or starch for these systems.

Another type of device that may be used to deliver carbapenems to the lung is a liquid spray device supplied, for example, by Aradigm Corporation (Hayward, Calif.). Liquid spray systems use extremely small nozzle holes to aerosolize liquid drug formulations that may then be directly inhaled into the lung.

In one embodiment, a nebulizer device is used to deliver carbapenems to the lung. Nebulizers create aerosols from liquid drug formulations by using, for example, ultrasonic energy or jet propulsion to form fine particles that may be readily inhaled (See e.g., Verschoyle et al., British J. Cancer, 1999, 80, Suppl 2, 96). Examples of nebulizers include devices supplied by Sheffield Pharmaceuticals, Inc. (Rochester, N.Y.), Aventis (Bridgewater, N.J.), BatellePharma (Columbus, Ohio), Aro Technologies, (Lagenthal, Switzerland), Omron Healthcare (Mt. Vernon, Ill.), DeVilbiss (Glendale Heights, Ill.) (Armer et al., U.S. Pat. No. 5,954,047; van der Linden et al., U.S. Pat. No. 5,950,619; van der Linden et al., U.S. Pat. No. 5,970,974) and include, but are not limited to, atomizing, jet, pressurized, vibrating or ultrasonic devices

In another embodiment, an electrohydrodynamic (“EHD”) aerosol device is used to deliver carbapenems to the lung. EHD aerosol devices use electrical energy to aerosolize liquid drug solutions or suspensions (see e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807). The electrochemical properties of the carbapenem formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of carbapenems are known to the skilled artisan and are within the scope of the invention.

### 4.6 Pharmaceutical Compositions

The present pharmaceutical compositions contain a therapeutically effective amount of one or more carbapenems or a pharmaceutically available salt, hydrate or solvate thereof, preferably, in purified form, together with a suitable amount of a pharmaceutically acceptable vehicle, so as to provide the form for proper administration to a patient. When administered to a patient, the carbapenem or a pharmaceutically available salt, hydrate or solvate thereof and the pharmaceutically acceptable vehicle are preferably sterile. Water is a preferred vehicle when the compounds of the invention are administered by inhalation. Saline solutions and aqueous dextrose and glycerol solutions can also be employed as liquid vehicles. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a carbapenem or a pharmaceutically available salt, hydrate or solvate thereof may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of carbapenems into preparations which can be used pharmaceutically. Proper formulation is dependent upon the route of administration chosen.

The present pharmaceutical compositions can take the form of solutions, suspensions, emulsions, aerosols, sprays, or any other form suitable for use. Liquid drug formulations suitable for use with nebulizers, liquid spray devices and EHD aerosol devices will typically include one or more carbapenems or a pharmaceutically available salt, hydrate or solvate thereof with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as an alcohol, glycol, polyglycol or a fatty acid. Other methods of formulating liquid drug solutions or suspensions suitable for use in aerosol devices are known to those of skill in the art (see, e.g., Biesalski, U.S. Pat. No. 5,112,598; Biesalski, U.S. Pat. No. 5,556,611).

Generally, a pharmaceutical composition suitable for delivery to the lung by nebulizers, liquid spray devices or EHD aerosol devices will contain a minimal amount of a carbapenem (e.g., between about 5 mg and about 2000 mg) or a pharmaceutically available salt, hydrate or solvate thereof formulated in the smallest possible volume of a pharmaceutically acceptable vehicle at an acceptable pH and preferably, with a certain degree of salinity. Preferably, the pharmaceutical composition will have a pH between about 4.5 and about 7.5, more preferably, between about 5.0 and about 7.0, most preferably, between about 5.0 and about 6.5. In some embodiments, the pharmaceutical composition will have a total osmolality between about 50 and about 550 mOsm/kg with a range of chloride or bicarbonate concentration between about 30 mM and about 300 mM.

In some embodiments, a pharmaceutical composition suitable for delivery to the lung will contain between about 5 mg to about 100 mg of a carbapenem or a pharmaceutically available salt, hydrate or solvate thereof dissolved between about 1 ml to about 5 ml of an aqueous solution with a pH between about 4.5 and about 7.5. Preferably, the pharmaceutical composition contains between at least about 30 mM chloride ion.

In other embodiments, a pharmaceutical composition suitable for delivery to the lung will contain between about 5 mg to about 100 mg of a carbapenem or a pharmaceutically available salt, hydrate or solvate thereof dissolved in about 1 ml to about 5 mL saline (preferably, normal saline diluted between about 4 fold and about 10 fold) at a pH of between about 5.0 and about 7.0. Preferably, this pharmaceutical composition will form aerosol particles having a predominant mass medium average diameter between about 1.0 μM and about 5.0 μM when nebulized by an atomizing, jet, electronic or ultrasonic nebulizer.

The present pharmaceutical compositions can also take the form of dry powders, prepared, for example, by lyophilization, precipitation or crystallization, which may be delivered to the lung, e.g., by a dry powder inhaler or a metered dose inhaler. Dry powder formulations are preferably between about 5 mg and about 2000 mg of a carbapenem or a pharmaceutically available salt, hydrate or solvate thereof, more preferably between about 20 mg and 1000 mg of a carbapenem or a pharmaceutically available salt, hydrate or solvate thereof. In some embodiments, a dry powder formulation comprises between about 20 mg and about 500 mg of doripenem or a pharmaceutically available salt, hydrate or solvate thereof. In other embodiments, a dry powder formulation comprises between about 50 mg and 300 mg of doripenem or a pharmaceutically available salt, hydrate or solvate thereof. The dry powder formulation may be milled, spray dried, precipitated, etc. to a powder having a predominant mass medium average diameter between about 1.0 μM and about 5.0 μM.

### 4.7 Doses

Carbapenems or pharmaceutical compositions thereof, will generally be used in an amount effective to achieve the intended purpose. For use to treat or prevent diseases or disorders characterized by pulmonary infection, carbapenems and/or pharmaceutical compositions thereof, are administered or applied in a therapeutically effective amount.

The amount of carbapenem that will be effective in the treatment of a particular disorder or condition disclosed herein will depend on the nature of the disorder or condition and can be determined by standard clinical techniques known in the art. In addition, in vitro or in vivo assays may optionally be employed to help identify optimal dosage ranges. The amount of a carbapenem administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration or by multiple applications. Dosing may be repeated intermittently, may be provided alone or in combination with other drugs and may continue as long as required for effective treatment of the disease state or disorder.

Suitable dosage ranges for administration by inhalation are dependent on the potency of the carbapenem, but are generally about 0.001 mg to about 200 mg of a carbapenem per kilogram body weight. Preferably, the carbapenem dosage is between about 5 mg and about 2000 mg, more preferably, between about 25 mg and about 500 mg. Preferably, a dose of doripenem is between about 20 mg and about 1000 mg, more preferably, between about 50 mg and about 300 mg. Dosage ranges may be readily determined by methods known to the artisan of ordinary skill.

Carbapenems are preferably assayed in vitro and in vivo, as described above, for the desired therapeutic or prophylactic activity, prior to use in humans. For example, in vitro assays can be used to determine whether administration of a specific carbapenem or a combination of carbapenems is preferred for treating pulmonary infections. Carbapenems may also be demonstrated to be effective and safe using animal model systems.

Preferably, a therapeutically effective dose of a carbapenems described herein will provide therapeutic benefit without causing substantial toxicity. Toxicity of carbapenems may be determined using standard pharmaceutical procedures and may be readily ascertained by the skilled artisan. The dose ratio between toxic and therapeutic effect is the therapeutic index. Carbapenems will preferably exhibit particularly high therapeutic indices in treating disease and disorders. The dosage of a carbapenems described herein will preferably an effective dose with little or no toxicity.

### 4.8 Combination Therapy

In certain embodiments, carbapenems and/or pharmaceutical compositions thereof can be used in combination therapy with at least one other therapeutic agent. The carbapenem and/or pharmaceutical composition thereof and the therapeutic agent can act additively or, more preferably, synergistically. In one embodiment, a carbapenem and/or pharmaceutical composition thereof is administered concurrently with the administration of another therapeutic agent, which may be part of the same pharmaceutical composition or a different pharmaceutical composition. In another embodiment, a carbapenem pharmaceutical composition is administered prior or subsequent to administration of another therapeutic agent (e.g., tobramycin, anti-inflammatory agents and mucolytic agents).

### 4.9 Therapeutic Kits

The current invention provides therapeutic kits comprising carbapenems or pharmaceutical compositions thereof. The therapeutic kits may also contain other compounds (e.g., other antibiotics, HIV inhibitors, etc.) or pharmaceutical compositions thereof.

Therapeutic kits may have a single container which contains a carbapenem or pharmaceutical compositions thereof with or without other components (e.g., other compounds or pharmaceutical compositions of these other compounds) or may have distinct container for each component. In one embodiment, a therapeutic kit includes a carbapenem or a pharmaceutical composition thereof packaged for use in combination with the co-administration of a second compound (preferably, another antibiotic) or a pharmaceutical composition thereof. The components of the kit may be pre-complexed or each component may be in a separate distinct container prior to administration to a patient.

The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container. In another embodiment, a therapeutic kit includes a carbapenem in one distinct container with a pharmaceutically acceptable vehicle in another container. Preferably, the carbapenem is doripenem and the pharmaceutically acceptable vehicle is saline

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

Preferably, a therapeutic kit will contain apparatus (e.g., one or more needles, syringes, eye droppers, pipette, electrohydrodynamic aerosol device, an aerosol device, a dry powder inhaler, a multi dose dry powder inhaler, a nebulizer device, etc.), which enables administration of the components of the kit.

In one embodiment, the therapeutic kit is a two-part reconstitution system such as a flexible medical container, which contains a selectively enlargeable container (e.g., Sperko et al., U.S. Pat. No. 6,468,377). In this embodiment, the carbapenem and diluent are stored separately until use.

### 5. EXAMPLES

The following example is provided by way of illustration only and not by way of limitation. Those of skill in the art will readily recognize a variety of noncritical parameters that could be changed or modified to yield essentially similar results.

### 5.1 Example 1

### Evaluation of Doripenem Delivery With Different Nebulizers

Bench testing (“in vitro”) using various nebulizers with doripenem solution was performed. Standard procedures for analyzing nebulizer performance were used.

The devices tested included the PARI LC PLUS (LC+, PARI GMbH) nebulizer (used to deliver TOBI® an FDA approved inhaled antibiotic), another jet nebulizer, the AeroEclipse (AE, Monaghan Medical Corp) and four other “new generation” devices such as the AeroNeb Pro (AN P, Aerogen, Inc.), the AeroNeb GO “Clinical” (AN Go, Aerogen, Inc.) the custom eFlow (PARI) and the Omron NE-U22 (MicroAir, Omron Corporation).

Doripenem was reconstituted with preservative free normal saline to a final concentration of 18 mg/mL. The fill volume (nebulizer charge) was 5 mL=90 mg for the LC+(same fill volume as TOBI®), and for the AN P. A smaller 3 mL=54 mg fill volume was used for the AE, AN, and eFlow since these devices were supposed to be more efficient than the PARI LC PLUS. The performance and properties of the technologies were rank-ordered at based on four desirable value-added properties expected by the cystic fibrosis community (Table 1). Size, portability, and noise were also rank-ordered based on manufacturer specifications with 1 being the best and 0.25 being the worst scores.

The Pari eFlow prototype performed significantly better than any of the other devices with respect to delivering a large quantities of highly respirable aerosol (lung dose) in the shortest period of time and being most efficient (least waste) of medication put into the nebulizer. This nebulizer provided more than twice the quantity of respirable doripenem aerosol per unit time than the second best device.

TABLE 1
Rank Ordering of Nebulizer Technologies

| | (Respirable) | Inferred dose | | | |
|---|---|---|---|---|---|
| | Output Rate (mg | fraction lost to | | | |
| | in <5 μm dia | Filter and/or | | Size Portability | Cumulative |
| | droplets/min) | Atmosphere | Efficiency | Noise | Weighted Score |
| PARI eFlow | 4.6 | 26% | 43% | 1 | 2.0 |
| AN Go | 1.6 | 53% | 19% | 1 | 1.0 |
| PARI LC+ | 1.7 | 23% | 11% | 0.5 | 0.9 |
| ADOR-01 An P | 1.8 | 12% | 20% | 0.25 | 0.9 |
| MicroAir | 0.9 | 52% | 16% | 1 | 0.8 |
| Aeroeclipse | 1.0 | 13% | 22% | 0.5 | 0.8 |
| Weight | 1 | 3 | 4 | 2 | |
| Value | Efficacy | Secondary | Medication | Compliance | |
| | Compliance | Exposure | Transfer Price | | |
| | | Compliance | | | |

### 5.2 Example 2

### Laser Particle Sizing of Doripenem Delivered by Nebulizer

A Malvern Spraytec laser was used for all measurements of doripenem delivered by the nebulizers of Example 1. The Spraytec software was configured to calculate and store the Dv10, Dv50, Dv90, the % of particles≦2μ, the % of particles≦5μ, and the calculated GSD (84.13%÷50%). Typically, the fine particle fraction of an aerosol is felt to be the size range that has the highest probability of depositing in the lower airways, defined as the proportion of particles≦5μ. However, the actual cutoff may be closer to 2μ or 3μ, so that analysis was also included (Smaldone, Respiratory Drug Delivery IX, 2004; Vol. 1: pp 179-186). The nebulizer was positioned so that the outlet was 2 cm from the center of the beam and as close to the receiving lens as the physical configuration of the individual nebulizer would allow. Each nebulizer was charged with drug, positioned as above, and run continuously for 2 minutes. Data collection commenced at the end of the first minute and continued for the duration of the second minute. Measurements were made and recorded every second so that at the end of recording period, 60 data points for each of the measured parameters were obtained. All 60 data points for each parameter were averaged and reported. Four units of each device were studied in duplicate and results for all 8 studies were averaged to determine the mean, standard deviation, and coefficient of variation. The results are shown in Table 2 below.

TABLE 2
Particle Sizing Results (Mean ± Std Dev, min-max value)

| | VMD | GSD | % ≦ 2μ | % ≦ 5μ |
|---|---|---|---|---|
| LC+ | 3.6 ± 0.1 | 2.4 ± 0.1 | 30.9% ± 1 | 63.6% ± 1 |
| | 3.4 − 3.8 | 2.3 − 2.5 | 28.8 − 32.7 | 61.5 − 65.7 |
| AN P | 5.3 ± 0.3 | 2.1 ± 0.03 | 20.6% ± 1 | 47.9% ± 2 |
| | 4.9 − 5.6 | 2.1 − 2.2 | 19.2 − 22.3 | 45.5 − 51.2 |
| eFlow | 4.0 ± 0.2 | 1.6 ± 0.1 | 18.3% ± 2 | 67.6% ± 4 |
| | 3.7 − 4.2 | 1.5 − 1.7 | 15.5 − 20.6 | 62.2 − 73.2 |
| AE | 3.5 ± 0.2 | 2.1 ± 0.1 | 28.6% ± 3 | 67.0% ± 2 |
| | 3.2 − 3.8 | 2.0 − 2.2 | 23.1 − 31.9 | 63.7 − 70.8 |
| AN Go | 5.5 ± 0.4 | 1.9 ± 0.02 | 16.3% ± 1 | 45.6% ± 4 |
| | 5.0 − 6.0 | 1.9 − 1.95 | 14.8 − 18.9 | 41.3 − 50.6 |
| Omron | 5.6 ± 0.2 | 1.8 ± 0.1 | 14.7% ± 3 | 43.4% ± 3 |
| MICROAIR | 5.4 − 6.0 | 1.7 − 2.0 | 11.1 − 19.3 | 38.7 − 46.7 |

### 5.3 Example 3

### Nebulizer Output

A PARI COMPAS breath simulator and Respirgard filters were used for all studies. The breath simulator was set to a rate=15, tidal volume=500 mL, I:E ratio=1:1, with a sinusoidal waveform (European Standard). These parameters were confirmed with a Cosmo2 breathing monitor (Novametrix Medical Systems, Inc.). All nebulizers were charged with 3 mL (54 mg), except the LC+(5 mL) and An P(5 mL) and were weighed dry, full, and at the end of each study to determine gravimetric output and residual volume. Nebulizers were connected to the inspiratory filters and breath simulator. Expiratory filters were used (and deposited drug assayed) with the eFlow, Aeroneb PRO, and Aeroneb GO devices. Each study was timed from the beginning of nebulization until the onset of sputter (AE, LC+) or until the end of nebulization. Each component (nebulizer, inspiratory filter and expiratory filter) was rinsed individually with a known quantity of distilled H₂O. The Aeroneb PRO was rinsed in its entirety along with all connectors and one way valves included per the ADOR-01 set-up. The samples were then measured with a Spectronic Genesys 5 spectrophotometer at 298λ to determine individual concentrations. The deposited drug mass was then calculated.

Direct measurements made included were:

Duration: Time in minutes from the beginning to the end of nebulization.
Residual Dose: Amount of drug remaining in the device at the end of nebulization.
Inspired Dose: Total amount of drug deposited on the inspiratory filter.
Expired Dose: Drug deposited on expiratory filters (when used).
   Calculations made included:
Fine Particle Dose (FPD): The inspired dose multiplied by the % of particles≦5μ.
Ultrafine Particle Dose (UFPD): The inspired dose multiplied by the % of particles≦2μ.
Output (FPD/min): The FPD÷duration.
Efficiency: Fine particle dose as a fraction of total loading dose.

The results are shown in Table 3 below.

TABLE 3
Breath Simulator Results (Mean ± Std Dev, min-max value)

| | Duration | Residual | Inspired | | UFPD | | FPD |
|---|---|---|---|---|---|---|---|
| | (min) | Dose (mg) | Dose (mg) | FPD (mg) | (mg) | FPD/min | Efficiency |
| LC+ | 9.4 ± 0.3 | 44.0 ± 2.6 | 25.2 ± 1.5 | 16.0 ± 0.9 | 7.8 ± 0.5 | 1.7 ± 0.1 | 17.8% ± 1 |
| | 9.2-9.9 | 39.6-46.9 | 23.0-27.3 | 14.6 -17.3 | 7.3-8.4 | 1.6-1.8 | 16.2-19.3 |
| AN P | 10.2 ± 1.6 | 41.7 ± 3.7 | 37.4 ± 2.5 | 17.9 ± 1.1 | 7.7 ± 0.5 | 1.8 ± 0.2 | 19.9% ± 1 |
| | 7.6-12.3 | 37.7-48.0 | 33.0-39.9 | 15.8-19.1 | 6.8-8.2 | 1.5-2.1 | 18.7-21.2 |
| eFlow | 5.1 ± 0.5 | 6.0 ± .9 | 34.1 ± 1.6 | 23.1 ± 1.0 | 6.3 ± 0.3 | 4.6 ± 0.5 | 42.7% ± 2 |
| | 4.4-5.8 | 3.9-6.8 | 30.8-36.4 | 20.8-24.6 | 6.1-6.7 | 3.9-5.1 | 38.5-45.6 |
| AE | 12.4 ± 0.7 | 29.2 ± 1.3 | 17.7 ± 1.0 | 11.8 ± 0.6 | 5.1 ± 0.3 | 1.0 ± 0.1 | 21.9% ± 1 |
| | 11.7-13.8 | 27.3-31.4 | 16.4-19.4 | 11.0-13.0 | 4.7-5.5 | 0.9-1.1 | 20.9-24.0 |
| AN Go | 6.9 ± 1.3 | 2.4 ± 0.5 | 22.9 ± 0.7 | 10.4 ± 0.3 | 3.8 ± 0.1 | 1.6 ± 0.2 | 19.3% ± 1 |
| | 5.5-8.8 | 1.9-3.2 | 21.9-23.6 | 10.0-10.8 | 3.6-3.9 | 1.2-1.9 | 18.5-19.9 |
| Omron | 11.1 ± 5.5 | 6.1 ± 1.1 | 19.7 ± 0.9 | 8.6 ± 0.4 | 2.9 ± 0.1 | 0.9 ± 0.4 | 15.9% ± 1 |
| MICROAIR | 5.7-22.7 | 4.9-7.8 | 18.8-21.1 | 8.1-9.2 | 2.8-3.1 | 0.4-1.5 | 15.1-16.7 |

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims. All publications and patents cited herein are incorporated by reference in their entirety.

1. A method of treating or preventing pulmonary infection in a patient comprising administering to a patient in need of such treatment or prevention a therapeutically effective amount of a carbapenem or a pharmaceutically acceptable salt, hydrate, solvate or pro-drug thereof.

2. The method of claim 1, wherein the patient has cystic fibrosis, pneumonia, ventilator associated pneumonia, bronchitis or bronchiectstasis.

3. The method of claim 1, wherein the carbapenem is imipenem, meropenem, faropenem, biapenem, ertapenem, panipenem, ritipenem or sulopenem.

4. The method of claim 1, wherein the carbapenem is a pyrrolidylthiocarbapenem of Formula (I): 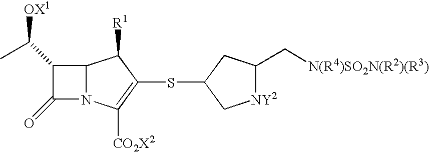
wherein:
R¹ is hydrogen or lower alkyl;
R², R³, R⁴ are independently hydrogen, lower alkyl, substituted lower alkyl, an amino protecting group or R² and R³ together with the nitrogen atom with which they are bonded form a saturated or unsaturated cyclic group or R² and R⁴ or R³ and R⁴ together with two nitrogen atoms and one sulfur atom in the sulfamide group form a saturated or unsaturated cyclic group; each cyclic group can further include at least one atom selected from the group consisting of oxygen, sulfur and nitrogen and each cyclic group can be substituted;
X¹ is hydrogen or a hydroxy protecting group;
X² is hydrogen, a carboxy protecting group, an ammonio group, an alkali metal or an alkaline earth metal; and
Y² is hydrogen or an amino protecting group.

5. The method of claim 4, wherein R¹ is methyl, R², R³, R⁴, X¹ and Y² are hydrogen and X² is hydrogen or an alkali metal.

6. The method of claim 1, wherein the therapeutically effective amount of the carbapenem or pharmaceutically acceptable salt, hydrate, solvate or pro-drug thereof is between about 5 mg and about 2000 mg.

7. The method of claim 1, wherein the therapeutically effective amount of carbapenem or pharmaceutically acceptable salt, hydrate, solvate or pro-drug thereof is between about 25 mg and about 500 mg.

8. The method of claim 5, wherein the therapeutically effective amount of doripenem or pharmaceutically acceptable salt, hydrate, solvate or pro-drug thereof is between about 20 mg and about 1000 mg.

9. The method of claim 5, wherein the therapeutically effective amount of doripenem or pharmaceutically acceptable salt, hydrate, solvate or pro-drug thereof is between about 50 mg and about300 mg.

10. The method of claim 1, wherein the therapeutically effective amount of carbapenem or pharmaceutically acceptable salt, hydrate, solvate or pro-drug thereof is administered by inhalation into the lung of the patient.

11. The method of claim 10, wherein the therapeutically effective amount of carbapenem or pharmaceutically acceptable salt, hydrate, solvate or pro-drug thereof is administered by an electrohydrodynamic aerosol device, an aerosol device a dry powder inhaler, a multi dose dry powder inhaler or a nebulizer device into the lung of the patient.

12. The method of claim 11, wherein the therapeutically effective amount of carbapenem or pharmaceutically acceptable salt, hydrate, solvate or pro-drug thereof is administered by a nebulizer device into the lung of the patient.

13. The method of claim 1 or claim 2, wherein the pulmonary infection is caused by both gram negative and gram positive bacteria.

14. The method of claim 1 or claim 2, wherein the pulmonary infection is caused by Psuedomonas aeruginosa, Staphylococcus aureaus, Haemophilus influenzae, Burkholderia cepacia, staphylococci Staphylococci (methicillin susceptible), Enterobacteriaceae, Moraxella catarrhalis, Bacteroides spp., Clostridium spp., Peptostreptococcus spp. and Neisseria spp.

15. A pharmaceutical composition suitable for treating a patient suffering from a pulmonary infection comprising an amount of a carbapenem or a pharmaceutically acceptable salt, hydrate, solvate or prodrug thereof and a pharmaceutically acceptable vehicle, said amount being sufficient to treat the pulmonary infection.

16. The pharmaceutical composition of claim 15, wherein the pharmaceutically acceptable vehicle is a liquid.

17. The pharmaceutical composition of claim 16, wherein the pharmaceutically acceptable vehicle is water, saline, alcohol, surfactant or perflourocarbon.

18. The pharmaceutical composition of claim 17, wherein the pharmaceutically acceptable vehicle is saline.

19. The pharmaceutical composition of claim 18, wherein between about 5 mg to about 250 mg of carbapenem or pharmaceutically acceptable salt, hydrate, solvate or prodrug thereof are dissolved in between about 1 to about 5 ml of between about a 0.1% and 0.9% saline solution.

20. The pharmaceutical composition of claim 19, wherein the pH is between about 4.5 to about 7.5.

21. The pharmaceutical composition of claim 19, wherein the pH is between about 5.5 to about 6.5.

22. A method of treating pulmonary infection in a patient comprising administering to a patient in need of such treatment the pharmaceutical composition of claim 15.

23. The method of claim 22, wherein the pharmaceutical composition is administered by inhalation into the lung of the patient.

24. The method of claim 23, wherein the pharmaceutical composition is administered by a nebulizer device into the lung of the patient.

25. The method of claim 24, wherein the nebulizer is a jet, electronic ultrasonic or atomization nebulizer.

26. The method of claim 24, wherein the pharmaceutical composition forms an aerosol of particles with mass median aerodynamic diameter of between about 1 micron to about 5 micron.

27. A pharmaceutical composition suitable for preventing pulmonary infection in a patient at risk of a pulmonary infection comprising administering to the patient an amount of a carbapenem or a pharmaceutically acceptable salt, hydrate, solvate or prodrug thereof and a pharmaceutically acceptable vehicle.

28. A method for preventing a pulmonary infection in a patient at risk of a pulmonary infection comprising administering to the patient the pharmaceutical composition of claim 15.

29. A therapeutic kit comprising:
a first container containing a carbapenem or pharmaceutically acceptable salt, hydrate, solvate or prodrug thereof as a dry powder; and
a second container containing a pharmaceutically acceptable vehicle.

30. The kit of claim 29, wherein the carbapenem is doripenem and the pharmaceutically acceptable vehicle is saline.
